(19) United States
(12) Patent Application Publication
(10) Pub. No.: US 2004/0220427 A1
(43) Pub. Date: Nov. 4, 2004

(54) Process for producing (meth)acrylic acid

(75) Inventors: Shuhei Yada, Mie (JP); Kenji Takasaki, Mie (JP); Yasushi Ogawa, Mie (JP); Yoshiro Suzuki, Mie (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: MITSUBISHI CHEMICAL CORPORATION, Tokyo (JP)

(21) Appl. No.: 10/853,199

(22) Filed: May 26, 2004

Related U.S. Application Data
(63) Continuation of application No. PCT/JP02/12331, filed on Nov. 26, 2002.

(30) Foreign Application Priority Data
Nov. 28, 2001 (JP) 2001-362898

(51) Int. Cl. C07C 51/16; C07C 51/42

(52) U.S. Cl. 562/545; 562/600

(57) ABSTRACT

An object of the invention is to provide a process in which Michael addition reaction products generated as by-products in a (meth)acrylic acid production step are pyrolyzed to enable high-purity (meth)acrylic acid to be recovered at a high recovery and troubles such as clogging in production steps are prevented.

The invention includes a (meth)acrylic acid-yielding reaction step in which a starting compound for (meth)acrylic acid production is subjected to an oxidation reaction, a distillation step in which light matters are separated from the liquid reaction mixture from the (meth)acrylic acid-yielding reaction step to obtain crude (meth)acrylic acid, a step in which by-products generated in the production steps are pyrolyzed to recover (meth)acrylic acid, and a step in which the (meth)acrylic acid recovered is supplied to the distillation step. The temperature for the pyrolysis of the by-products is from 140 to 240° C. and the pressure for the pyrolysis is from 70 to 130 kPa.

### TECHNICAL FIELD

The present invention relates to a process for producing (meth)acrylic acid. More particularly, the invention relates to a process for (meth)acrylic acid production which includes a step in which by-products of (meth)acrylic acid production are pyrolyzed to recover (meth)acrylic acid, etc.

Incidentally, the term (meth)acrylic acid in this description is a general term for acrylic acid and methacrylic acid, and it may be either of these or may be both.

### BACKGROUND ART

As is generally known, reactions for yielding acrylic acid include the vapor-phase oxidation of propylene. Methods for this propylene oxidation for obtaining acrylic acid include a two-stage oxidation process in which oxidation to acrolein and subsequent oxidation to acrylic acid are conducted in separate reactors because these oxidation reactions differ in conditions and a process in which the starting material is oxidized directly to acrylic acid through one-stage oxidation.

FIG. 3 is a flow diagram in which acrylic acid is yielded by two-stage oxidation. Propylene, water vapor, and air pass through a first reactor and a second reactor which are packed with a molybdenum catalyst or the like, and the propylene is thus oxidized in two steps to give an acrylic acid-containing gas. This acrylic acid-containing gas is brought into contact with water in a collection column (condensation column) to obtain an aqueous acrylic acid solution. An appropriate extractant is added to this solution. Extraction is conducted in an extraction column, and the extractant is separated in a solvent separation column. Subsequently, acetic acid is separated in an acetic acid separation column to obtain crude acrylic acid. By-products are separated from this crude acrylic acid in a rectifier, whereby purified acrylic acid is obtained.

In place of the solvent extraction method in which acrylic acid is recovered from the aqueous acrylic acid solution with an extractant, an azeotropic separation method is also employed recently which comprises distilling the solution using water and an entrainer to obtain as a distillate an azeotropic mixture comprising water and the entrainer through the top of the azeotropic separation column and recover acrylic acid through the bottom of the column, as in FIGS. 1 and 2 which will be described later.

In the case of methacrylic acid, isobutylene or t-butyl alcohol is used in place of propylene. This starting material undergoes the same oxidation process to give purified methacrylic acid.

Fractions obtained by separating the crude acrylic acid and crude methacrylic acid through distillation/purification contain useful by-products including Michael addition products. These are hence decomposed to recover acrylic acid, etc.

JP-B-61-35977 describes a method in which Michael addition products are subjected to a decomposition reaction with a film evaporator and, simultaneously therewith, the decomposition reaction products are distilled off.

JP-A-11-12222 describes a method in which Michael addition products contained in acrylic acid are pyrolyzed to obtain acrylic acid and this acrylic acid is returned to a purification column for recovering acrylic acid as a distillate.

In the method in which Michael addition reaction products in by-products generated in a (meth)acrylic acid production step are decomposed to recover (meth)acrylic acid, there are cases where light impurities generated by the decomposition come into the recovered (meth)acrylic acid to contaminate it, resulting in an increased impurity concentration in the product (meth)acrylic acid. Furthermore, there are cases where impurities precipitate in a (meth)acrylic acid production step to form a cause of the inhibition of continuous operation.

When the decomposition reaction of a Michael addition product is conducted by the reaction distillation technique described in JP-B-61-35977, which employs a film evaporator and uses conditions including a decomposition temperature of 180° C. and a pressure of from 200 to 250 mmHg, then the acrylic acid recovered contains maleic anhydride which has comes thereinto in a high concentration. Even when this acrylic acid is recycled to a purification system, the maleic anhydride accumulates in the system without being discarded and, during this operation, the maleic anhydride partly changes into maleic acid and further isomerizes to fumaric acid. These acids are causative of troubles such as a decrease in flowability in the bottom's lines and solid precipitation when operational conditions fluctuate in some degree, and such troubles may prevent a stable continuous operation. Furthermore, in case where reaction distillation conditions under which maleic anhydride is recovered in a high concentration into the acrylic acid recovered, the residue comes to have an increased viscosity and impaired flowability in the discharge piping, making a continuous operation difficult.

It has been further found that when the decomposition reaction of a Michael addition product is conducted by the liquid-phase pyrolysis technique described in JP-A-11-12222 and the acrylic acid recovered is sent to an acrylic acid rectifier (a column for recovering acrylic acid as a distillate through the top), this process has a drawback that the product has increased concentrations of acrolein, acetic acid, and water and impaired quality, besides the above-described problem concerning maleic anhydride.

An object of the invention is to overcome the problems described above and provide a process for producing (meth)acrylic acid which includes the step of pyrolyzing by-products generated in a (meth)acrylic acid production step, including Michael addition reaction products, to recover (meth)acrylic acid and which can prevent impurities from coming into product (meth)acrylic acid and produce high-quality (meth)acrylic acid.

### DISCLOSURE OF THE INVENTION

The process for producing (meth)acrylic acid of the invention comprises a (meth)acrylic acid-yielding reaction step in which a starting compound for (meth)acrylic acid production is subjected to an oxidation reaction, a distillation step in which light matters are separated from the liquid reaction mixture from the (meth)acrylic acid-yielding reaction step to obtain crude (meth)acrylic acid, and a step in which by-products generated in the production steps are pyrolyzed to recover (meth)acrylic acid, characterized in that the (meth)acrylic acid recovered is supplied to the distillation step.

As stated above, the (meth)acrylic acid recovered through the pyrolysis of by-products including Michael addition products generated in a (meth)acrylic acid production step contains light impurities. According to the invention, the decomposition products are returned to the distillation step for light-matter separation in the (meth)acrylic acid production step, whereby light impurities can be prevented from coming into product (meth)acrylic acid.

### BRIEF DESCRIPTION OF THE DRAWINGS

FIG. 1 is a flow diagram of acrylic acid production according to the invention.

FIG. 2 is another flow diagram of acrylic acid production according to the invention.

FIG. 3 is a flow diagram of acrylic acid production according to a related-art technique.

### BEST MODE FOR CARRYING OUT THE INVENTION

The invention will be explained below in more detail.

In the invention, (meth)acrylic acid is preferably produced through: a reaction step in which propane, propylene, (meth)acrolein, isobutylene, t-butyl alcohol, or the like is subjected to a catalytic vapor-phase oxidation reaction; a distillation step in which (meth)acrylic acid is separated from low-boiling compounds such as water and acetic acid to obtain crude (meth)acrylic acid; a rectification step in which the crude (meth)acrylic acid is rectified; and a pyrolysis step in which by-products generated in the rectification step are pyrolyzed.

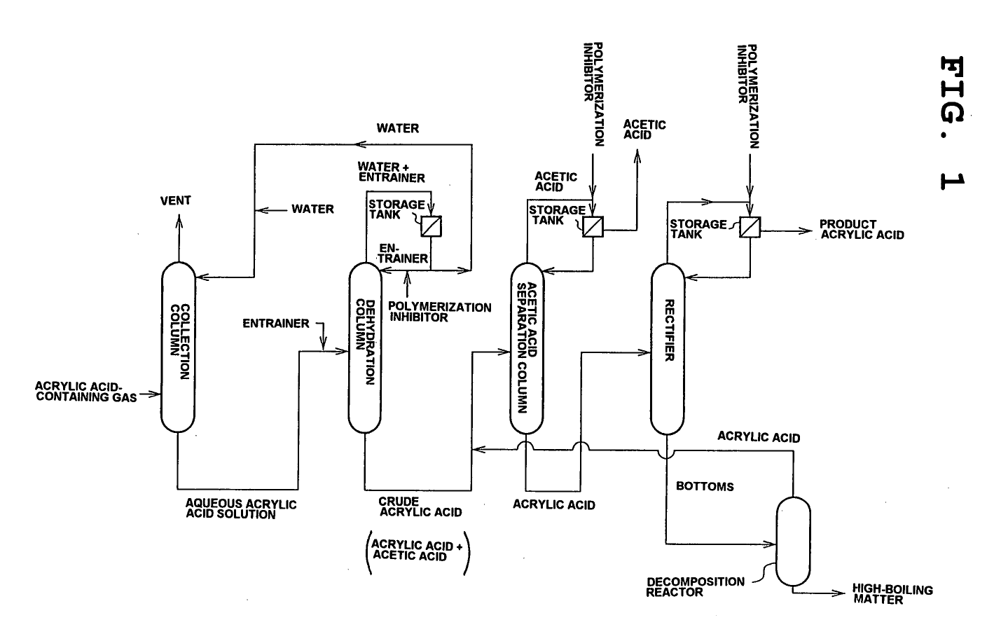

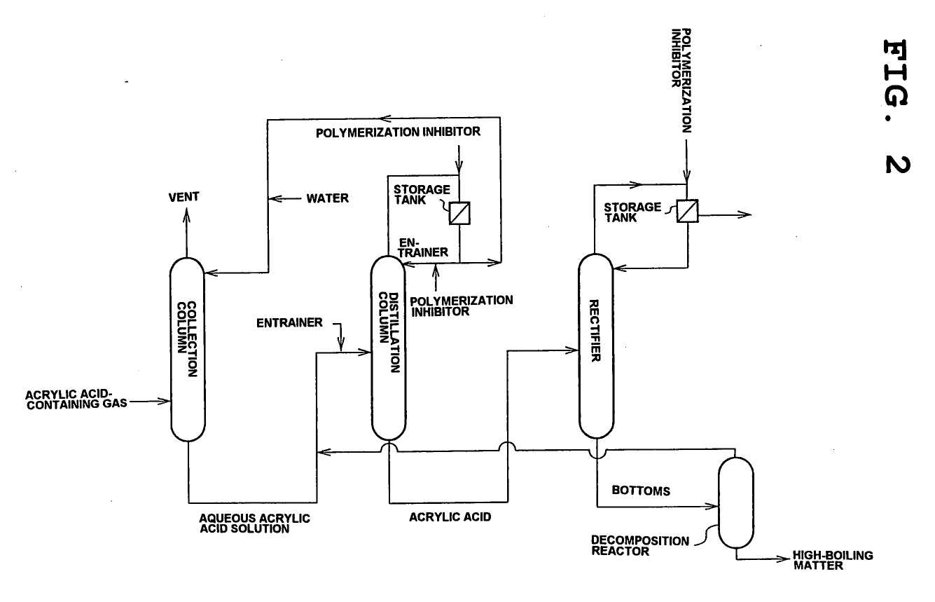

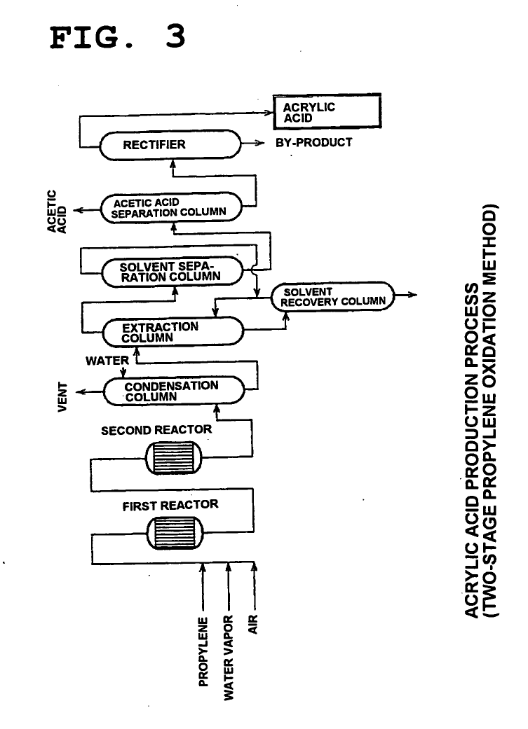

The separation of water from (meth)acrylic acid is accomplished, for example, by subjecting an aqueous (meth)acrylic acid solution obtained by quenching with water the gaseous oxidation reaction products yielded in the reaction step to separation by the azeotropic distillation method using an entrainer (FIG. 1) or separation by the extraction method using a solvent (FIG. 3). Incidentally, acetic acid may be separated after water separation, or water and acetic acid may be simultaneously separated with an entrainer (FIG. 2).

Michael addition products are by-products which generate in a (meth)acrylic acid production step, oxidation reaction, and subsequent various purification steps, especially on the bottom of each distillation column having a relatively high temperature, and are compounds formed by the Michael addition of (meth)acrylic acid, acetic acid, or water to a compound having a (meth)acryloyl group. Examples of the compound having a (meth)acryloyl group include (meth)acrolein and (meth)acrylic acid, and further include compounds to which (meth)acrylic acid has bonded by the Michael addition. Namely, examples thereof include carboxylic acids having an acryloyl group, such as the β-acryloxypropionic acid or β-methacryloxyisobutyric acid (hereinafter, dimer) formed by the Michael addition of (meth)acrylic acid to (meth)acrylic acid, a (meth)acrylic acid trimer (hereinafter, trimer) formed by the Michael addition of (meth)acrylic acid to the dimer, and a (meth)acrylic acid tetramer (hereinafter, tetramer) formed by the Michael addition of (meth)acrylic acid to the trimer. Examples thereof further include aldehydes having a (meth)acryloyl group likewise formed by the Michael addition of (meth)acrylic acid to (meth)acrolein. Specific examples of other Michael addition products include β-acetoxypropionic acid, β-hydroxypropionic acid, the β-acetoxy-substituted forms and β-hydroxy-substituted forms of the dimer, trimer, tetramer, and the like, β-acetoxypropanal, β-hydroxypropanal, aldehydes formed by the Michael addition of acetic acid or water to aldehydes formed by the Michael addition of (meth)acrylic acid to (meth)acrolein, and the like. These aldehydes are present also in the form of an acetal although the abundance ratio between these varies depending on the environment in which these are present in the process.

Such Michael addition products accumulate in a high concentration in the bottoms of a rectifier for obtaining purified (meth)acrylic acid from crude (meth)acrylic acid. It is hence preferred to pyrolyze the bottoms from the rectifier to recover (meth)acrylic acid. Although the bottoms from the rectifier contain the Michael addition products in a high concentration, they further contain acrylic acid and heavy matters such as the polymerization inhibitor used in the process and oligomers and polymers generated in the process.

In the invention, the decomposition reaction of a Michael addition product can be conducted by any of the continuous process, batch process, semi-batch process, intermittent-discharge process, and the like. However, the continuous process is preferred. The type of the reactor also is not particularly limited, and a reactor of any type can be employed, such as a complete mixing type stirring vessel reactor, circulating complete-mixing vessel reactor, reactor having a mere cavity, or the like. From the standpoint of enabling the decomposition reaction to be conducted by the reaction distillation technique, use may be made of either a reactor having a distillation column and condenser connected to an upper part thereof or a reactor which has been united with such apparatus, i.e., a distillation column, condenser, and reboiler.

The temperature for the decomposition reaction is preferably from 140 to 240° C., especially from 160 to 200° C. The liquid residence time as calculated from the amount of the liquid discharged is preferably from 0.2 to 50 hours, especially from 0.5 to 2 hours. In the case where the decomposition reaction is conducted continuously, the liquid residence time calculated from the amount of the liquid discharged can be regarded as the reaction time. For example, when the liquid capacity of the reactor is 500 L and the liquid discharge amount is 100 L/H, then the residence time is 5 hours.

The operating pressure is preferably from 70 to 130 kPa. It is, however, preferred to use an operating pressure higher than the vapor pressure of maleic anhydride at the reaction temperature. For example, when the reaction is conducted at 200° C., it is preferred to carry out the reaction at an operating pressure of 96 kPa or higher for avoiding the accumulation of maleic anhydride.

The distillation residue resulting from the reaction distillation is withdrawn for use as a fuel, etc. The distillate obtained by the reactor distillation is supplied to a distillation column which is a light-matter separation column in the (meth)acrylic acid purification step and serves to separate low-boiling ingredients, such as (meth)acrolein, acetic acid, and water, through the column top. In the case of FIG. 3 and FIG. 1, which will be explained below, the distillate is supplied to an acetic acid separation column. In the case of FIG. 2, the distillate is supplied to a distillation column for simultaneously separating water and acetic acid. The light-matter separation column may be an azeoptroic distillation column for separating water.

FIG. 1 is a flowchart of an acrylic acid production step in which an aqueous acrylic acid solution is separated into acrylic acid and water by distillation and acetic acid is then separated by distillation.

The acrylic acid-containing gas obtained by the catalytic vapor-phase oxidation of propylene and/or acrolein with a gas containing molecular oxygen is introduced into an acrylic acid collection column and contacted with water to give an aqueous acrylic acid solution.

Incidentally, the acrylic acid-containing gas further contains N₂, CO₂, acetic acid, water, etc. Part of the acetic acid and the N₂ and CO₂ are withdrawn as a bent gas through the top of the collection column.

The aqueous acrylic acid solution from this collection column is supplied to a dehydration column together with an entrainer. An azeotropic mixture comprising water and the entrainer is obtained as a distillate through the top of the column, while acrylic acid containing acetic acid is obtained through the column bottom. The azeotropic mixture which comprises water and the entrainer and has been obtained as a distillate through the top of the dehydration column is introduced into a storage tank, where the mixture is separated into an organic phase consisting mainly of the entrainer and an aqueous phase consisting mainly of water. The organic phase is circulated to the dehydration column after a polymerization inhibitor is added thereto. On the other hand, the aqueous phase is circulated to the acrylic acid collection column and used as collection water to be contacted with the acrylic acid-containing gas. According to need, water is replenished through a water return line. Furthermore, for the purpose of recovering the entrainer from the water in the water return line, the water may be passed through an entrainer recovery column (not shown) before being circulated to the acrylic acid collection column.

The crude acrylic acid discharged through the bottom of the dehydration column is introduced into an acetic acid separation column in order to remove the acetic acid remaining therein. The acetic acid is separated and removed through the top of the column. There are cases where the acetic acid recovered through the column top is partly returned to the process because it contains acrylic acid.

Through the bottom of the acetic acid separation column, acrylic acid containing substantially no acetic acid is obtained. This acrylic acid is introduced into a rectifier, where high-boiling matters are separated and removed to give high-purity acrylic acid as a product. The bottoms (high-boiling matters) from the rectifier are introduced into a decomposition reactor. Products of the decomposition reaction which include acrylic acid are supplied to the acetic acid separation column.

FIG. 2 is a flow sheet showing a process for acrylic acid production in which the functions of the dehydration column and acetic acid separation column in FIG. 1 have been united in a distillation column.

An aqueous acrylic acid solution from a collection column is introduced into the distillation column after an entrainer is added thereto. Water, acetic acid, and an azeotrope are obtained as distillates through the top of the distillation column. The azeotrope is returned to the distillation column, while the water and the acetic acid are returned to the collection column. The acetic acid is discharged from the system as a collection column vent gas. The treatment flowchart for the bottoms from the distillation column is the same as the treatment flowchart for the bottoms from the acetic acid separation column in FIG. 1. The acrylic acid and others from the decomposition reactor are returned to the distillation column.

### EXAMPLES

The invention will be explained below in detail by reference to Example and Comparative Examples.

### Example 1

The invention was practiced according to the acrylic acid production step shown in FIG. 2. Namely, bottoms from the rectifier in the acrylic acid production step shown in FIG. 2 were pyrolyzed in the decomposition reactor, and products of the decomposition were supplied to the azeotropic distillation column.

The bottoms from the rectifier for separating heavy matters from acrylic acid had a composition comprising 21% by weight acrylic acid, 7.9% by weight maleic anhydride, 1.0% by weight β-hydroxypropionic acid, 51.1% by weight β-acryloxypropionic acid, 2% by weight acrylic acid trimer, 1.5% by weight β-acetoxypropionic acid, and 15% by weight heavy matters and others. The bottoms were fed to the decomposition reactor at 22 kg/h. The decomposition reactor was a stirring vessel made of Hastelloy C having an inner diameter of 200 mm and a height of 400 mm. A distillation column having an inner diameter of 30 mm and a height of 1,000 mm and packed with a coil packing to 500 mm and the attached condenser were connected to an upper part of the reactor. A heat medium was supplied to the external jacket of the reactor to regulate the reaction temperature to 190° C., and the liquid level in the decomposition reactor was regulated so that the liquid residence time as calculated from liquid discharge amount became 1 hour. The reaction pressure was kept at 100 kPa. The operation could be stably continued over 70 hours without arousing pipe clogging or other troubles. A distillate was obtained from the decomposition reactor at 16 kg/h on the average. The composition thereof was analyzed by gas chromatography and, as a result, found to consist of 90.4% by weight acrylic acid, 3.9% by weight maleic anhydride, 2.5% by weight β-acryloxypropionic acid, 1.5% by weight other heavy matters, and light matters consisting of 0.49% by weight acrolein, 0.29% by weight water, and 0.93% by weight acetic acid. A residue was obtained at 6 kg/h on the average, and the composition thereof was analyzed by gas chromatography and, as a result, found to consist of 10.5% by weight acrylic acid, 15.7% by weight maleic anhydride, 18.3% by weight β-acryloxypropionic acid, and 55.5% by weight heavy matters and others. The product acrylic acid had undergone no decrease in purity.

### Comparative Example 1

A decomposition reaction was conducted using the same feed material, same experimental apparatus, and same conditions as in Example 1, except that the decomposition reaction temperature was 180° C. and the pressure was 27 kPa.

In a 70-hour continuous operation, a trouble that the residue discharge line was slightly clogged occurred twice, but a by-pass line was used to continue the operation. A distillate was obtained at 18 kg/h on the average. The composition of the distillate was analyzed by gas chromatography and, as a result, found to consist of 81.0% by weight acrylic acid, 7.5% by weight maleic anhydride, 7.0% by weight β-acryloxypropionic acid, 3.0% by weight other heavy matters, and light matters consisting of 0.44% by weight acrolein, 0.26% by weight water, and 0.83% by weight acetic acid. A residue was obtained at 4 kg/h on the average, and the composition thereof was analyzed by gas chromatography and, as a result, found to consist of 9.8% by weight acrylic acid, 5.9% by weight maleic anhydride, 19.6% by weight β-acryloxypropionic acid, and 64.7% by weight heavy matters and others.

### Comparative Example 2

The same procedure as in Example 1 was conducted, except that the distillate from the decomposition reactor was supplied to the acrylic acid rectifier. As a result, the concentrations of the water, acrolein, and acetic acid which came into the product acrylic acid increased to 160 ppm, 260 ppm, and 490 ppm, respectively. Such impurity levels were not on an allowable level.

While the invention has been described in detail and with reference to specific embodiments thereof, it will be apparent to one skilled in the art that various changes and modifications can be made therein without departing from the spirit and scope thereof.

This application is based on a Japanese patent application filed on Nov. 28, 2001 (application No. 2001-362898), the entire contents thereof being hereby incorporated by reference.

<Industrial Applicability>

As described above, according to the invention, Michael addition reaction products generated as by-products in a (meth)acrylic acid production step are pyrolyzed, whereby (meth)acrylic acid having a high purity can be recovered at a high recovery. Furthermore, according to the invention, troubles in production steps, such as the clogging caused especially by a maleic acid compound, can be prevented to enable a continuous operation to be conducted stably.

1. A process for producing (meth)acrylic acid which comprises
a (meth)acrylic acid-yielding reaction step in which a starting compound for (meth)acrylic acid production is subjected to an oxidation reaction,
a distillation step in which light matters are separated from the liquid reaction mixture from the (meth)acrylic acid-yielding reaction step to obtain crude (meth)acrylic acid,
a step in which by-products generated in the reaction step and distillation step are pyrolyzed to recover (meth)acrylic acid, and
a step in which the (meth)acrylic acid recovered is supplied to the distillation step, characterized in that
the temperature for the pyrolysis of the by-products is from 140 to 240° C. and
the pressure for the pyrolysis is from 70 to 130 kPa.

2. The process for producing (meth)acrylic acid as claimed in claim 1, characterized in that
the process comprises a rectification step in which the crude (meth)acrylic acid obtained in the distillation step is rectified and
the by-products are bottoms from a rectifier in the rectification step.

3. The process for producing (meth)acrylic acid as claimed in claim 1, characterized in that the by-products comprise Michael addition products.

4. The process for producing (meth)acrylic acid as claimed in claim 3, characterized in that the Michael addition product is a compound formed by the addition of water, acetic acid, or (meth)acrylic acid to the acryloyl group of (meth)acrylic acid.
